(19) United States
(12) Patent Application Publication
(10) Pub. No.: US 2025/0095829 A1
(43) Pub. Date: Mar. 20, 2025

(54) LEARNING DEVICE, TRAINED MODEL, MEDICAL DIAGNOSTIC DEVICE, ULTRASOUND ENDOSCOPE DEVICE, LEARNING METHOD, AND PROGRAM

(71) Applicant: FUJIFILM CORPORATION, Tokyo (JP)

(72) Inventor: Toshihiro USUDA, Kanagawa (JP)

(21) Appl. No.: 18/964,700

(22) Filed: Dec. 2, 2024

Related U.S. Application Data
(63) Continuation of application No. PCT/JP2023/021602, filed on Jun. 9, 2023.

(30) Foreign Application Priority Data
Jun. 29, 2022 (JP) 2022-105153

(51) Int. Cl. G16H 30/40; G06T 3/60; G06T 7/00; G06V 20/70

(52) U.S. Cl. CPC G16H 30/40; G06T 3/60; G06T 7/0014; G06V 20/70; G06T 2207/10068; G06T 2207/10132; G06T 2207/20081; G06T 2207/30096

(57) ABSTRACT

A learning device includes a first processor. The first processor acquires a plurality of medical images to which annotations for specifying a lesion are assigned, and trains a model using the plurality of acquired medical images. The medical image is an image that is generated based on at least one convex ultrasound image and that has an aspect imitating at least a part of a radial ultrasound image.

### CROSS-REFERENCE TO RELATED APPLICATIONS

This application is a continuation application of International Application No. PCT/JP2023/021602, filed Jun. 9, 2023, the disclosure of which is incorporated herein by reference in its entirety. Further, this application claims priority from Japanese Patent Application No. 2022-105153, filed Jun. 29, 2022, the disclosure of which is incorporated herein by reference in its entirety.

### BACKGROUND

### 1. Technical Field

The technology of the present disclosure relates to a learning device, a trained model, a medical diagnostic device, an ultrasound endoscope device, a learning method, and a program.

### 2. Related Art

JP1997-084793A (JP-H09-084793A) discloses an ultrasound image processing device. In the ultrasound image processing device disclosed in JP1997-084793A (JP-H09-084793A), an ultrasound probe control unit drives an ultrasound probe so as to execute three-dimensional scanning of a subject by combining radial scanning and linear scanning. In the ultrasound image processing device disclosed in JP1997-084793A (JP-H09-084793A), an ultrasound observation device sequentially creates a plurality of consecutive ultrasound tomographic images from echo signals of the subject obtained by the three-dimensional scanning using the ultrasound probe via the ultrasound probe control unit, and sequentially outputs the plurality of consecutive ultrasound tomographic images to a tomographic image monitor. The tomographic image monitor sequentially displays the ultrasound tomographic images.

WO2022/071326A discloses an information processing device. The information processing device disclosed in WO2022/071326A comprises an image acquisition unit and a first position information output unit. The image acquisition unit acquires a catheter image obtained by a radial scanning type image acquisition catheter. The first position information output unit inputs the acquired catheter image to a medical instrument trained model that receives input of the catheter image and that outputs first position information related to a position of a medical instrument included in the catheter image, and outputs the first position information.

JP2000-316864A discloses an ultrasound diagnostic device. The ultrasound diagnostic device disclosed in JP2000-316864A includes an ultrasound observation device that transmits and receives ultrasound and that obtains a real-time echo image (ultrasound tomographic image), and an image processing device that executes various types of image processing based on echo data obtained by the ultrasound observation device.

### SUMMARY

One embodiment according to the technology of the present disclosure provides a learning device, a trained model, a medical diagnostic device, an ultrasound endoscope device, a learning method, and a program that can contribute to specifying a lesion shown in a radial ultrasound image via a trained model without using a trained model obtained by training a model only using the radial ultrasound image.

A first aspect according to the technology of the present disclosure relates to a learning device comprising: a first processor, in which the first processor acquires a plurality of medical images to which annotations for specifying a lesion are assigned, and trains a model using the plurality of acquired medical images, and the medical image is an image that is generated based on at least one convex ultrasound image and that has an aspect imitating at least a part of a radial ultrasound image.

A second aspect according to the technology of the present disclosure relates to the learning device according to the first aspect, in which the plurality of medical images include a circular image generated by combining a plurality of the convex ultrasound images.

A third aspect according to the technology of the present disclosure relates to the learning device according to the second aspect, in which a scale of the circular image is adjusted based on a scale of the radial ultrasound image.

A fourth aspect according to the technology of the present disclosure relates to the learning device according to the second or third aspect, in which the circular image is stored in a first memory in advance, and the first processor acquires the circular image from the first memory, and trains the model using the acquired circular image.

A fifth aspect according to the technology of the present disclosure relates to the learning device according to any one of the first to fourth aspects, in which the plurality of medical images include a rotated image obtained by rotating the convex ultrasound image.

A sixth aspect according to the technology of the present disclosure relates to the learning device according to the fifth aspect, in which a scale of the rotated image is adjusted based on a scale of the radial ultrasound image.

A seventh aspect according to the technology of the present disclosure relates to the learning device according to the fifth or sixth aspect, in which the rotated image is stored in a second memory in advance, and the first processor acquires the rotated image from the second memory, and trains the model using the acquired rotated image.

An eighth aspect according to the technology of the present disclosure relates to the learning device according to any one of the first to seventh aspects, in which the plurality of medical images include a scale-adjusted image obtained by adjusting a scale of the convex ultrasound image based on a scale of the radial ultrasound image.

A ninth aspect according to the technology of the present disclosure relates to the learning device according to the eighth aspect, in which the scale-adjusted image is stored in a third memory in advance, and the first processor acquires the scale-adjusted image from the third memory, and trains the model using the acquired scale-adjusted image.

A tenth aspect according to the technology of the present disclosure relates to the learning device according to the first aspect, in which the first processor randomly selects one generation method from among a plurality of generation methods for generating the medical image based on the at least one convex ultrasound image, acquires the medical image by generating the medical image in accordance with the selected generation method, and trains the model using the acquired medical image.

An eleventh aspect according to the technology of the present disclosure relates to the learning device according to the tenth aspect, in which the plurality of generation methods include a first generation method, a second generation method, and a third generation method, the first generation method includes generating a circular image as the medical image by combining a plurality of the convex ultrasound images, the second generation method includes generating a rotated image in which the convex ultrasound image is rotated, as the medical image, and the third generation method includes generating a scale-adjusted image as the medical image by adjusting a scale of the convex ultrasound image based on a scale of the radial ultrasound image.

A twelfth aspect according to the technology of the present disclosure relates to the learning device according to the eleventh aspect, in which the first generation method includes adjusting a scale of the circular image based on the scale of the radial ultrasound image.

A thirteenth aspect according to the technology of the present disclosure relates to the learning device according to the eleventh or twelfth aspect, in which the second generation method includes adjusting a scale of the rotated image based on the scale of the radial ultrasound image.

A fourteenth aspect according to the technology of the present disclosure relates to the learning device according to any one of the first to thirteenth aspects, in which the first processor acquires at least one first ultrasound image obtained by a first radial ultrasound endoscope, and trains the model using the acquired first ultrasound image.

A fifteenth aspect according to the technology of the present disclosure relates to the learning device according to any one of the first to fourteenth aspects, in which the first processor acquires a virtual ultrasound image that is generated based on volume data showing a subject and that has an aspect imitating at least a part of the radial ultrasound image, and trains the model using the acquired virtual ultrasound image.

A sixteenth aspect according to the technology of the present disclosure relates to a trained model obtained by training the model using the plurality of medical images via the learning device according to any one of the first to fifteenth aspects.

A seventeenth aspect according to the technology of the present disclosure relates to a trained model comprising: a data structure used for processing of specifying a lesion from a second ultrasound image obtained by a second radial ultrasound endoscope, in which the data structure is obtained by training a model using a plurality of medical images to which annotations for specifying the lesion are assigned, and the medical image is an image that is generated based on at least one convex ultrasound image and that has an aspect imitating at least a part of a radial ultrasound image.

An eighteenth aspect according to the technology of the present disclosure relates to a medical diagnostic device comprising: the trained model according to the sixteenth or seventeenth aspect; and a second processor, in which the second processor acquires a third ultrasound image obtained by a third radial ultrasound endoscope, and detects a portion corresponding to the lesion from the acquired third ultrasound image in accordance with the trained model.

A nineteenth aspect according to the technology of the present disclosure relates to an ultrasound endoscope device comprising: the trained model according to the sixteenth or seventeenth aspect; a fourth radial ultrasound endoscope; and a third processor, in which the third processor acquires a fourth ultrasound image obtained by the fourth radial ultrasound endoscope, and detects a portion corresponding to the lesion from the acquired fourth ultrasound image in accordance with the trained model.

A twentieth aspect according to the technology of the present disclosure relates to a learning method comprising: acquiring a plurality of medical images to which annotations for specifying a lesion are assigned; and training a model using the plurality of acquired medical images, in which the medical image is an image that is generated based on at least one convex ultrasound image and that has an aspect imitating at least a part of a radial ultrasound image.

A twenty-first aspect according to the technology of the present disclosure relates to a program for causing a computer to execute a process comprising: acquiring a plurality of medical images to which annotations for specifying a lesion are assigned; and training a model using the plurality of acquired medical images, in which the medical image is an image that is generated based on at least one convex ultrasound image and that has an aspect imitating at least a part of a radial ultrasound image.

### BRIEF DESCRIPTION OF THE DRAWINGS

Exemplary embodiments of the technology of the disclosure will be described in detail based on the following figures, wherein:

FIG. 1 is a conceptual diagram showing an example of an aspect in which an endoscope system is used;

FIG. 2 is a conceptual diagram showing an example of an overall configuration of the endoscope system;

FIG. 3 is a block diagram showing an example of a configuration of an ultrasound endoscope device;

FIG. 4 is a block diagram showing an example of a configuration of a learning device;

FIG. 5 is a conceptual diagram showing an example of processing contents of an acquisition unit and a learning execution unit of the learning device;

FIG. 6 is a conceptual diagram showing an example of a method of creating a circular image, a scale-adjusted image, a rotated image, a radial ultrasound image, and a virtual image used to train an NN;

FIG. 7 is a conceptual diagram showing an example of processing contents of a generation unit, a detection unit, and a control unit of a processing device;

FIG. 8 is a flowchart showing an example of a flow of learning execution processing;

FIG. 9 is a flowchart showing an example of a flow of lesion detection processing;

FIG. 10 is a conceptual diagram showing an example of a processing content of an acquisition unit according to a first modification example; and

FIG. 11 is a flowchart showing an example of a flow of learning execution processing according to the first modification example.

### DETAILED DESCRIPTION

Hereinafter, an example of embodiments of a learning device, a trained model, a medical diagnostic device, an ultrasound endoscope device, a learning method, and a program according to the technology of the present disclosure will be described with reference to the accompanying drawings.

First, the terms used in the following description will be described.

CPU is an abbreviation for “central processing unit”. GPU is an abbreviation for “graphics processing unit”. TPU is an abbreviation for “tensor processing unit”. RAM is an abbreviation for “random-access memory”. NVM is an abbreviation for “non-volatile memory”. EEPROM is an abbreviation for “electrically erasable programmable read-only memory”. ASIC is an abbreviation for “application-specific integrated circuit”. PLD is an abbreviation for “programmable logic device”. FPGA is an abbreviation for “field-programmable gate array”. SoC is an abbreviation for “system-on-a-chip”. SSD is an abbreviation for “solid-state drive”. USB is an abbreviation for “Universal Serial Bus”. HDD is an abbreviation for “hard disk drive”. EL is an abbreviation for “electro-luminescence”. CMOS is an abbreviation for “complementary metal-oxide-semiconductor”. CCD is an abbreviation for “charge-coupled device”. CT is an abbreviation for “computed tomography”. MRI is an abbreviation for “magnetic resonance imaging”. PC is an abbreviation for “personal computer”. LAN is an abbreviation for “local area network”. WAN is an abbreviation for “wide area network”. AI is an abbreviation for “artificial intelligence”. BLI is an abbreviation for “blue light imaging”. LCI is an abbreviation for “linked color imaging”. NN is an abbreviation for “neural network”. CNN is an abbreviation for “convolutional neural network”. R-CNN is an abbreviation for “region-based convolutional neural network”. YOLO is an abbreviation for “you only look once”. RNN is an abbreviation for “recurrent neural network”. FCN is an abbreviation for “fully convolutional network”.

In the present embodiment, the term “match” means the match in the sense of including an error generally allowed in the technical field to which the technology of the present disclosure belongs, that is, an error to the extent that it does not contradict the gist of the technology of the present disclosure, in addition to the exact match. In the present embodiment, the term “same” means the same in the sense of including an error generally allowed in the technical field to which the technology of the present disclosure belongs, that is, an error to the extent that it does not contradict the gist of the technology of the present disclosure, in addition to the exact same. In the present embodiment, a radial ultrasound image means an ultrasound image obtained by a radial scanning type ultrasound endoscopy. In the present embodiment, a convex ultrasound image means an ultrasound image obtained by a convex scanning type ultrasound endoscopy. In addition, hereinafter, it is assumed that a scale of the radial ultrasound image is smaller than a scale of the convex ultrasound image.

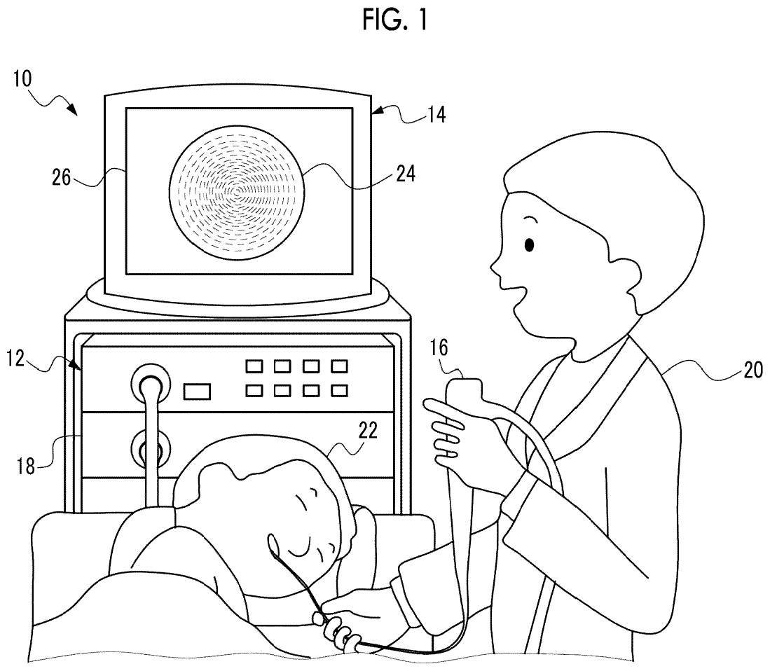

As shown in FIG. 1 as an example, an endoscope system 10 comprises an ultrasound endoscope device 12 and a display device 14. The ultrasound endoscope device 12 comprises a radial ultrasound endoscope 16 (hereinafter, referred to as an “ultrasound endoscope 16”) and a processing device 18. The ultrasound endoscope device 12 is an example of a “medical diagnostic device” and an “ultrasound endoscope device” according to the technology of the present disclosure. The ultrasound endoscope 16 is an example of a “first radial ultrasound endoscope”, a “second radial ultrasound endoscope”, a “third radial ultrasound endoscope”, and a “fourth radial ultrasound endoscope” according to the technology of the present disclosure.

The ultrasound endoscope 16 is a radial scanning type ultrasound endoscope. The ultrasound endoscope device 12 is used by a doctor 20 or the like. The processing device 18 is connected to the ultrasound endoscope 16, and transmits and receives various signals to and from the ultrasound endoscope 16. That is, the processing device 18 outputs the signal to the ultrasound endoscope 16 to control an operation of the ultrasound endoscope 16, or executes various types of signal processing on the signal input from the ultrasound endoscope 16.

The ultrasound endoscope device 12 is a device for executing medical care (for example, diagnosis and/or treatment) on a medical care part (for example, an organ such as a pancreas) in a body of a subject 22, and generates and outputs an ultrasound image indicating an observation target region including the medical care target part.

For example, in a case of observing the observation target region in the body of the subject 22, the doctor 20 inserts the ultrasound endoscope 16 into the body of the subject 22 from a mouth or a nose of the subject 22 (in the example shown in FIG. 1, the mouth), and emits ultrasound at a position such as a stomach or a duodenum. Since the ultrasound endoscope 16 is the radial scanning type ultrasound endoscope, the ultrasound endoscope 16 emits the ultrasound in a concentric pattern and detects a reflected wave obtained by reflecting the emitted ultrasound in the observation target region.

It should be noted that the example shown in FIG. 1 shows an aspect in which an upper gastrointestinal endoscopy is executed, but the technology of the present disclosure is not limited to this, and the technology of the present disclosure can also be applied to a lower gastrointestinal endoscopy, a bronchoscopy, and the like. That is, the technology of the present disclosure can be applied as long as a radial scanning type ultrasound endoscopy is executed.

The processing device 18 generates, in a specific image mode, an ultrasound image 24 based on the reflected wave detected by the ultrasound endoscope 16 and outputs a radial ultrasound image 24 to the display device 14 or the like. The specific image mode is a brightness mode (B-mode). However, the B-mode is merely an example, and an amplitude mode (A-mode), a motion mode (M-mode), or the like may be used.

The radial ultrasound image 24 is an ultrasound image having a circular outer shape. The radial ultrasound image 24 is a moving image including a plurality of frames generated at a specific frame rate (for example, several tens of frames/second). Here, the moving image is described as an example, but this is merely an example, and the technology of the present disclosure is implementable even in a case in which the radial ultrasound image 24 is a still image. The radial ultrasound image 24 is an example of a “second ultrasound image”, a “third ultrasound image”, and a “fourth ultrasound image” according to the technology of the present disclosure.

It should be noted that, hereinafter, for convenience of description, in a case in which it is not necessary to distinguish between the radial ultrasound image 24 obtained by the ultrasound endoscope device 12 in a radial ultrasound endoscopy on the subject 22 and the other radial ultrasound images, the radial ultrasound image 24 and the other radial ultrasound images will be referred to as a “radial ultrasound image” without reference numerals. Here, the other radial ultrasound images refer to, for example, radial ultrasound images obtained in the specific image mode (here, as an example, the B-mode) by one or more radial ultrasound endoscopies (for example, one or more radial ultrasound endoscopies executed earlier than the radial ultrasound endoscopy shown in FIG. 1) on one or more subjects other than the subject 22. In the other radial ultrasound images, an observation target region corresponding to the observation target region shown in the radial ultrasound image 24 is shown.

The display device 14 displays various types of information including an image under the control of the processing device 18. Examples of the display device 14 include a liquid-crystal display and an EL display. The radial ultrasound image 24 generated by the processing device 18 is displayed as the moving image on a screen 26 of the display device 14. The doctor 20 observes the radial ultrasound image 24 displayed on the screen 26 to determine whether or not a lesion is shown in the observation target region, and specifies, in a case in which the lesion is found, a position of the lesion in the observation target region through the radial ultrasound image 24.

It should be noted that the example shown in FIG. 1 shows a form example in which the radial ultrasound image 24 is displayed on the screen 26 of the display device 14, but this is merely an example, and the radial ultrasound image 24 may be displayed on a display device (for example, a display of a tablet terminal) other than the display device 14. The radial ultrasound image 24 may be stored in a computer-readable non-transitory storage medium (for example, a flash memory, an HDD, and/or a magnetic tape).

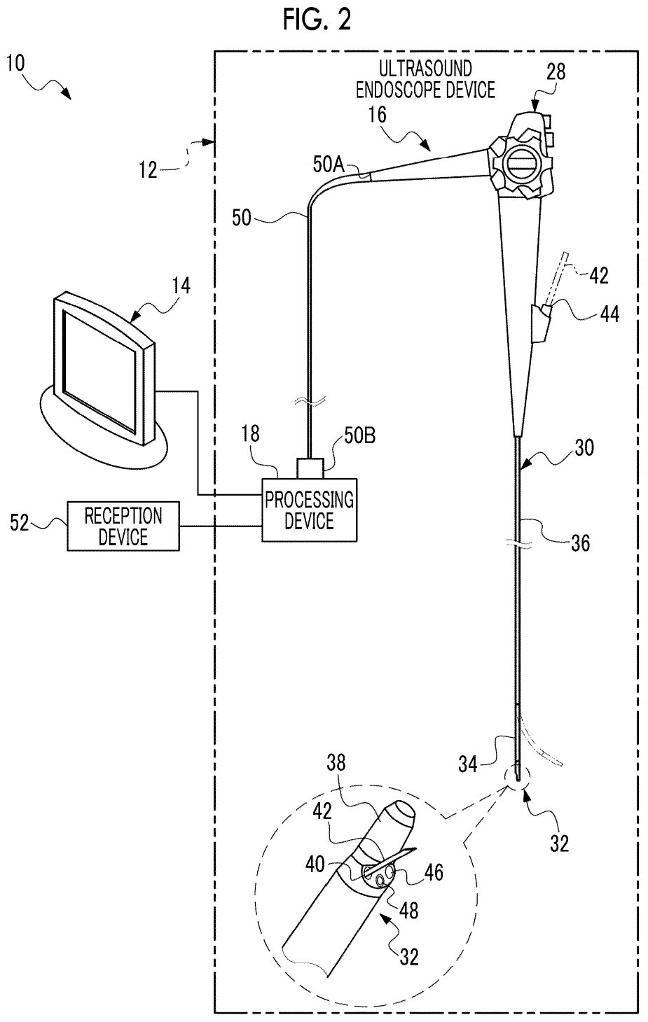

As shown in FIG. 2 as an example, the ultrasound endoscope 16 comprises an operating part 28 and an insertion part 30. The insertion part 30 is formed in a tubular shape. The insertion part 30 includes a distal end part 32, a bendable part 34, and a flexible part 36. The distal end part 32, the bendable part 34, and the flexible part 36 are disposed in an order of the distal end part 32, the bendable part 34, and the flexible part 36 from a distal end side to a base end side of the insertion part 30. The flexible part 36 is formed of a material having a long and flexible shape and connects the operating part 28 and the bendable part 34. The bendable part 34 is partially bent or rotated about an axial center of the insertion part 30 by operating the operating part 28. As a result, the insertion part 30 is sent to a back side of a luminal organ while being bent in accordance with a shape of the luminal organ (for example, a shape of a duodenal pathway) or being rotated about an axis of the insertion part 30.

The distal end part 32 is provided with an ultrasound probe 38 and a treatment tool opening 40. The ultrasound probe 38 is provided on the distal end side of the distal end part 32. The ultrasound probe 38 is formed in a cylindrical shape, and emits the ultrasound in a concentric pattern with respect to an axis of the ultrasound probe 38 and receives the reflected wave obtained by the emitted ultrasound being reflected in the observation target region.

The treatment tool opening 40 is formed on the base end side of the distal end part 32 with respect to the ultrasound probe 38. The treatment tool opening 40 is an opening for allowing a treatment tool 42 to protrude from the distal end part 32. A treatment tool insertion port 44 is formed at the operating part 28, and the treatment tool 42 is inserted into the insertion part 30 through the treatment tool insertion port 44. The treatment tool 42 passes through the insertion part 30 and protrudes from the treatment tool opening 40 to the outside of the ultrasound endoscope 16. The treatment tool opening 40 also functions as a suction port for suctioning blood, internal waste, and the like.

In the example shown in FIG. 2, a puncture needle is shown as the treatment tool 42. It should be noted that this is merely an example, and the treatment tool 42 may be forceps and/or a sheath.

In the example shown in FIG. 2, an illumination device 46 and a camera 48 are provided in the distal end part 32. The illumination device 46 emits light. Examples of a type of light emitted from the illumination device 46 include visible light (for example, white light), invisible light (for example, near-infrared light), and/or special light. Examples of the special light include light for BLI and/or light for LCI.

The camera 48 images the inside of the luminal organ using an optical method. Examples of the camera 48 include a CMOS camera. The CMOS camera is merely an example, and another type of camera, such as a CCD camera, may be used. It should be noted that the image obtained by being captured by the camera 48 is displayed on the display device 14, is displayed on a display device (for example, a display of a tablet terminal) other than the display device 14, or is stored in a storage medium (for example, a flash memory, an HDD, and/or a magnetic tape).

The ultrasound endoscope device 12 comprises the processing device 18 and a universal cord 50. The universal cord 50 has a base end part 50A and a distal end part 50B. The base end part 50A is connected to the operating part 28. The distal end part 50B is connected to the processing device 18.

The endoscope system 10 comprises a reception device 52. The reception device 52 is connected to the processing device 18. The reception device 52 receives an instruction from a user. Examples of the reception device 52 include an operation panel having a plurality of hard keys and/or a touch panel, a keyboard, a mouse, a trackball, a foot switch, a smart device, and/or a microphone.

The processing device 18 executes various types of signal processing or transmits and receives various signals to and from the ultrasound endoscope 16 or the like in response to the instruction received by the reception device 52. For example, the processing device 18 emits the ultrasound to the ultrasound probe 38 in response to the instruction received by the reception device 52, generates the radial ultrasound image 24 (see FIG. 1) based on the reflected wave received by the ultrasound probe 38, and outputs the generated radial ultrasound image 24.

The display device 14 is also connected to the processing device 18. The processing device 18 controls the display device 14 in response to the instruction received by the reception device 52. As a result, for example, the radial ultrasound image 24 generated by the processing device 18 is displayed on the screen 26 of the display device 14 (see FIG. 1).

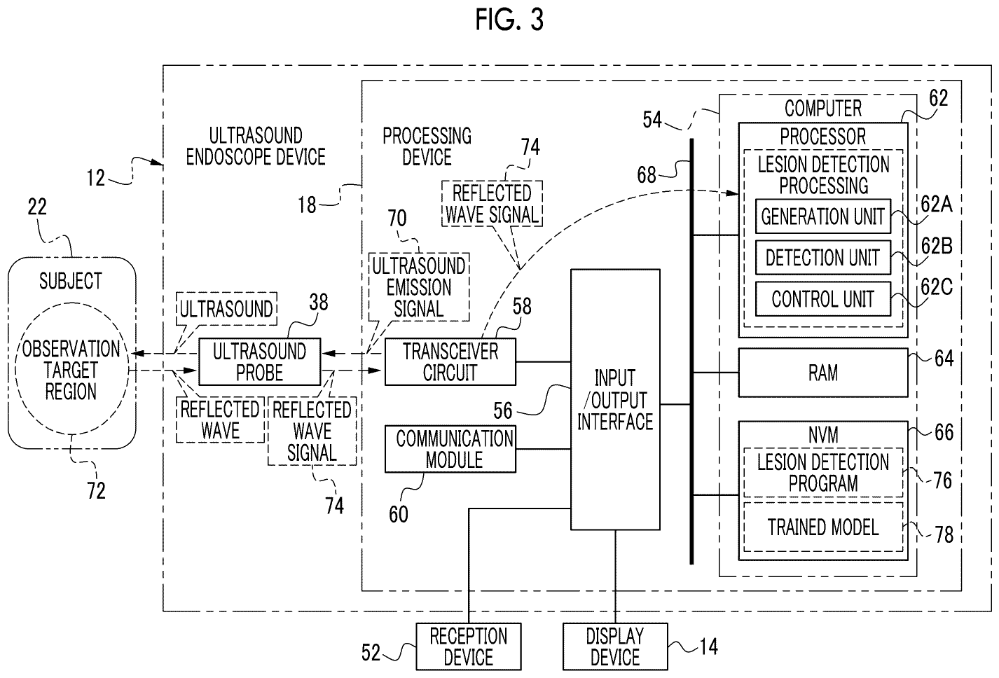

As shown in FIG. 3 as an example, the processing device 18 comprises a computer 54, an input/output interface 56, a transceiver circuit 58, and a communication module 60.

The computer 54 comprises a processor 62, a RAM 64, and an NVM 66. The input/output interface 56, the processor 62, the RAM 64, and the NVM 66 are connected to a bus 68.

The processor 62 controls the entire processing device 18. For example, the processor 62 includes a CPU and a GPU, and the GPU is operated under the control of the CPU, and is mainly responsible for executing image processing. It should be noted that the processor 62 may be one or more CPUs integrated with a GPU function or may be one or more CPUs not integrated with the GPU function. The processor 62 may include a multi-core CPU or a TPU. The processor 62 is an example of a “second processor” and a “third processor” according to the technology of the present disclosure.

The RAM 64 is a memory that temporarily stores information, and is used as a work memory by the processor 62. The NVM 66 is a non-volatile storage device that stores various programs and various parameters. Examples of the NVM 66 include a flash memory (for example, an EEPROM) and/or an SSD. It should be noted that the flash memory and the SSD are merely examples, and the NVM 66 may be other non-volatile storage devices such as an HDD, or may be a combination of two or more types of non-volatile storage devices.

The reception device 52 is connected to the input/output interface 56, and the processor 62 acquires the instruction received by the reception device 52 via the input/output interface 56 and executes processing in response to the acquired instruction.

The transceiver circuit 58 is connected to the input/output interface 56. The transceiver circuit 58 generates an ultrasound emission signal 70 having a pulse waveform to output the ultrasound emission signal 70 to the ultrasound probe 38 in response to an instruction from the processor 62. The ultrasound probe 38 converts the ultrasound emission signal 70 input from the transceiver circuit 58 into the ultrasound and emits the ultrasound to the observation target region 72 of the subject 22. The ultrasound is emitted from the ultrasound probe 38 in a concentric pattern. The ultrasound probe 38 receives the reflected wave obtained by reflecting the ultrasound emitted from the ultrasound probe 38 from the observation target region 72, converts the reflected wave into a reflected wave signal 74, which is an electric signal, and outputs the reflected wave signal 74 to the transceiver circuit 58. The transceiver circuit 58 digitizes the reflected wave signal 74 input from the ultrasound probe 38 and outputs the digitized reflected wave signal 74 to the processor 62 via the input/output interface 56. The processor 62 generates the radial ultrasound image 24 (see FIG. 1) as the ultrasound image showing an aspect of a tomographic plane of the observation target region 72 based on the reflected wave signal 74 input from the transceiver circuit 58 via the input/output interface 56.

Although not shown in FIG. 3, the illumination device 46 (see FIG. 2) is also connected to the input/output interface 56. The processor 62 controls the illumination device 46 via the input/output interface 56 to change the type of the light emitted from the illumination device 46 or to adjust an amount of the light. In addition, although not shown in FIG. 3, the camera 48 (see FIG. 2) is also connected to the input/output interface 56. The processor 62 controls the camera 48 via the input/output interface 56 or acquires the image obtained by imaging the inside of the body of the subject 22 using the camera 48 via the input/output interface 56.

The communication module 60 is connected to the input/output interface 56. The communication module 60 is an interface including a communication processor, an antenna, and the like. The communication module 60 is connected to a network (not shown) such as a LAN or a WAN, and controls communication between the processor 62 and an external device.

The display device 14 is connected to the input/output interface 56, and the processor 62 controls the display device 14 via the input/output interface 56 such that various types of information are displayed on the display device 14.

The reception device 52 is connected to the input/output interface 56, and the processor 62 acquires the instruction received by the reception device 52 via the input/output interface 56 and executes processing in response to the acquired instruction.

The NVM 66 stores a lesion detection program 76 and a trained model 78. The processor 62 executes lesion detection processing by reading out the lesion detection program 76 from the NVM 66 and executing the readout lesion detection program 76 on the RAM 64. The lesion detection processing is processing of detecting the lesion from the observation target region 72 using an AI method. The processor 62 executes the lesion detection processing to detect the lesion from the observation target region 72 by detecting a portion corresponding to the lesion from the radial ultrasound image 24 (see FIG. 1) in accordance with the trained model 78. The lesion detection processing is implemented by the processor 62 operating as a generation unit 62A, a detection unit 62B, and a control unit 62C in accordance with the lesion detection program 76 executed on the RAM 64.

It should be noted that the trained model 78 is a trained model having a data structure used for processing of specifying the lesion from the radial ultrasound image. The trained model 78 is an example of a “trained model” according to the technology of the present disclosure.

The trained model 78 is an NN used to detect the portion corresponding to the lesion from the radial ultrasound image 24. Therefore, in order to obtain the trained model 78, the radial ultrasound image generated by the radial scanning type ultrasound endoscope is ideal as the ultrasound image to be learned by an NN that has not been trained.

However, at present, the number of radial ultrasound endoscopies executed is significantly smaller than the number of convex ultrasound endoscopies executed. Therefore, it is difficult to collect the radial ultrasound images used to train the NN, in a number required to obtain the target detection accuracy.

On the other hand, the number of convex ultrasound endoscopies executed is significantly larger than the number of radial ultrasound endoscopies executed. This means that the number of convex ultrasound images generated by convex ultrasound endoscopies is significantly larger than the number of radial ultrasound images. That is, the convex ultrasound image can be more easily collected than the radial ultrasound image.

Therefore, in the present embodiment, an image that is generated based on the convex ultrasound image and that has an aspect imitating at least a part of the radial ultrasound image is used as a training ultrasound image for obtaining the trained model 78. Hereinafter, details will be described.

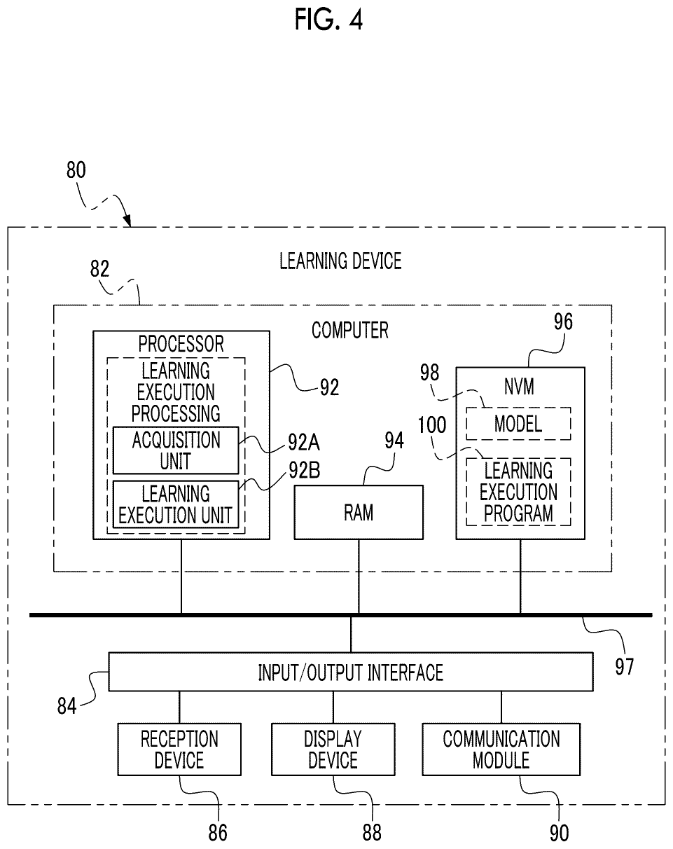

As shown in FIG. 4 as an example, a learning device 80 comprises a computer 82, an input/output interface 84, a reception device 86, a display device 88, and a communication module 90. The computer 82 comprises a processor 92, a RAM 94, and an NVM 96. The input/output interface 84, the processor 92, the RAM 94, and the NVM 96 are connected to a bus 97. The learning device 80 is an example of a “learning device” according to the technology of the present disclosure. The computer 82 is an example of a “computer” according to the technology of the present disclosure. The processor 92 is an example of a “first processor” according to the technology of the present disclosure. The NVM 96 is an example of a “first memory”, a “second memory”, and a “third memory” according to the technology of the present disclosure.

It should be noted that, since a plurality of hardware resources (that is, the processor 92, the RAM 94, and the NVM 96) included in the computer 82 shown in FIG. 4 are the same type as a plurality of hardware resources included in the computer 54 shown in FIG. 3, the description of duplicate parts will be omitted. In addition, the input/output interface 84 shown in FIG. 4 is the same as the input/output interface 56 shown in FIG. 3, the reception device 86 shown in FIG. 4 is the same as the reception device 52 shown in FIG. 3, the display device 88 shown in FIG. 4 is the same as the display device 14 shown in FIG. 3, and the communication module 90 shown in FIG. 4 is the same as the communication module 60 shown in FIG. 3, and thus the description thereof will be omitted here.

The NVM 96 stores a model 98 that has not been trained and a learning execution program 100. An example of the model 98 is a mathematical model using the NN. Examples of a type of the NN include a YOLO, an R-CNN, and an FCN. In addition, the NN used in the model 98 may be, for example, the YOLO, the R-CNN, or a combination of the FCN and an RNN. The RNN is suitable for learning of a plurality of images obtained in time series. It should be noted that the type of the NN described here is merely an example, and other types of the NNs capable of detecting an object by learning an image may be used.

The processor 92 controls the entire learning device 80. The processor 92 executes learning execution processing by reading out the learning execution program 100 from the NVM 96 and executing the readout learning execution program 100 on the RAM 94. The learning execution processing is processing of creating the trained model 78 (see FIG. 3) by training the model 98 using training data. The learning execution processing is implemented by the processor 92 operating as an acquisition unit 92A and a learning execution unit 92B in accordance with the learning execution program 100 executed on the RAM 94.

It should be noted that the model 98 is an example of a “model” according to the technology of the present disclosure. The learning execution program 100 is an example of a “program” according to the technology of the present disclosure. The learning execution processing is an example of “processing” according to the technology of the present disclosure.

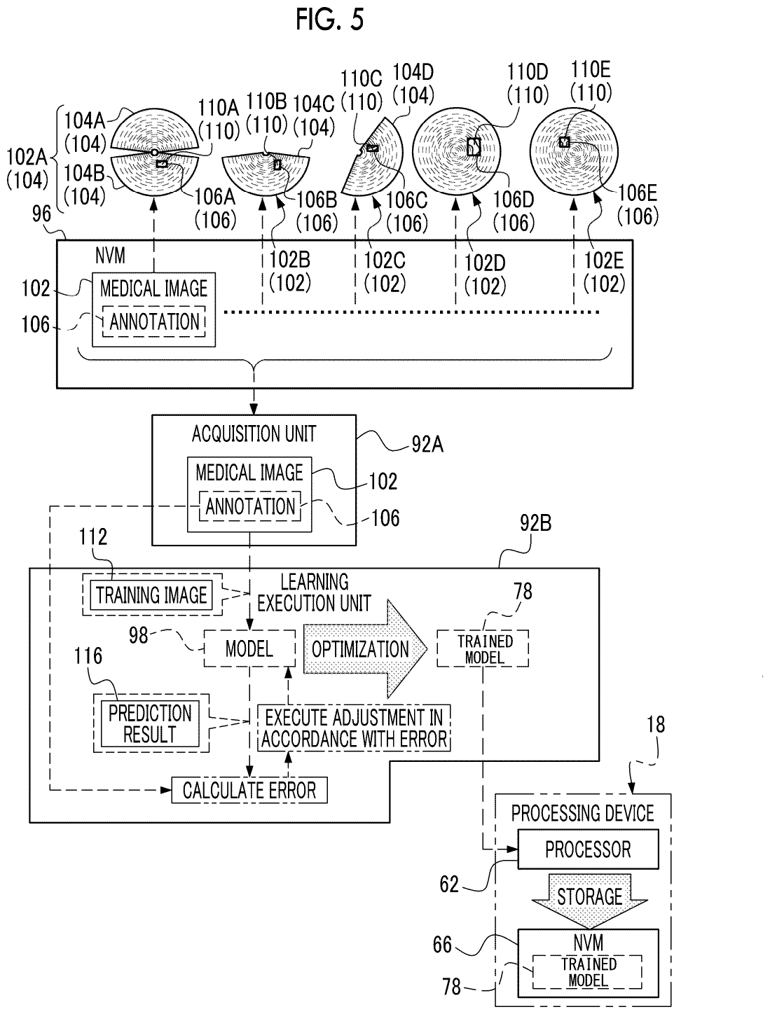

As an example, as shown in FIG. 5, the NVM 96 stores a plurality of medical images 102 in advance. The plurality of medical images 102 are images obtained from a plurality of subjects (for example, a plurality of subjects other than the subject 22 shown in FIG. 1 or a plurality of subjects including the subject 22). The plurality of medical images 102 include the image that is generated based on at least one convex ultrasound image 104 and that has an aspect imitating at least a part of the radial ultrasound image. The convex ultrasound image 104 is an ultrasound image obtained in the same image mode (here, as an example, the B-mode) as the radial ultrasound image 24 shown in FIG. 1.

The types of the medical images 102 are roughly classified into five types of a circular image 102A, a scale-adjusted image 102B, a rotated image 102C, a radial ultrasound image 102D, and a virtual image 102E. In the NVM 96, a plurality of different circular images 102A, a plurality of different scale-adjusted images 102B, a plurality of different rotated images 102C, a plurality of different radial ultrasound images 102D, and a plurality of different virtual images 102E are stored as the medical images 102. The circular image 102A, the scale-adjusted image 102B, and the rotated image 102C are images that are generated based on at least one convex ultrasound image 104 and that have an aspect imitating at least a part of the radial ultrasound image. The image that has an aspect imitating at least a part of the radial ultrasound image refers to, for example, an image having a shape closer to the radial ultrasound image than to the convex ultrasound image itself that is obtained from the convex ultrasound endoscopy and that is not processed at all and/or an image obtained by adjusting the convex ultrasound image to a scale close to or the same as the scale of the radial ultrasound image.

It should be noted that, in the present embodiment, the circular image 102A, the scale-adjusted image 102B, and the rotated image 102C are examples of a “medical image” according to the technology of the present disclosure. The circular image 102A is an example of a “circular image” according to the technology of the present disclosure. The scale-adjusted image 102B is an example of a “scale-adjusted image” according to the technology of the present disclosure. The rotated image 102C is an example of a “rotated image” according to the technology of the present disclosure. The radial ultrasound image 102D is an example of a “first ultrasound image” according to the technology of the present disclosure. The virtual image 102E is an example of a “virtual ultrasound image” according to the technology of the present disclosure.

As will be described in detail later, the circular image 102A is an image that is generated based on convex ultrasound images 104A and 104B. An outer shape of the circular image 102A need not be a perfect circular shape and may be an incomplete circular shape. The incomplete circular shape refers to, for example, a shape closer to the outer shape (that is, a circular shape) of the radial ultrasound image than to the outer shape (that is, a fan shape) of the convex ultrasound image itself that is obtained by the convex ultrasound endoscopy and that is not processed at all. Examples of the incomplete circular shape include a circular shape (for example, a circular shape with a part cut out) in which a gap is partially formed as shown in FIG. 5.

The lesion is shown in the circular image 102A. That is, the circular image 102A includes a lesion region 110A that is the portion corresponding to the lesion. An annotation 106A is assigned to the circular image 102A. The annotation 106A is information capable of specifying a position of the lesion region 110A in the circular image 102A (for example, information including a plurality of coordinates capable of specifying a position of a rectangular frame that circumscribes the lesion region 110A).

Here, for convenience of description, as an example of the annotation 106A, the information capable of specifying the position of the lesion region 110A in the circular image 102A is shown, but this is merely an example. For example, the annotation 106A may include other types of information capable of specifying the lesion shown in the circular image 102A, such as information capable of specifying a type of the lesion shown in the circular image 102A.

As will be described in detail later, the scale-adjusted image 102B is an image that is generated based on a convex ultrasound image 104C. The lesion is shown in the scale-adjusted image 102B. That is, the scale-adjusted image 102B includes a lesion region 110B that is the portion corresponding to the lesion. An annotation 106B is assigned to the scale-adjusted image 102B. The annotation 106B is information capable of specifying a position of the lesion region 110B in the scale-adjusted image 102B (for example, information including a plurality of coordinates capable of specifying a position of a rectangular frame that circumscribes the lesion region 110B).

Here, for convenience of description, as an example of the annotation 106B, the information capable of specifying the position of the lesion region 110B in the scale-adjusted image 102B is shown, but this is merely an example. For example, the annotation 106B may include other types of information capable of specifying the lesion shown in the scale-adjusted image 102B, such as information capable of specifying a type of the lesion shown in the scale-adjusted image 102B.

As will be described in detail later, the rotated image 102C is an image that is generated based on a convex ultrasound image 104D. The lesion is shown in the rotated image 102C. That is, the rotated image 102C includes a lesion region 110C that is the portion corresponding to the lesion. An annotation 106C is assigned to the rotated image 102C. The annotation 106C is information capable of specifying a position of the lesion region 110C in the rotated image 102C (for example, information including a plurality of coordinates capable of specifying a position of a rectangular frame that circumscribes the lesion region 110C).

Here, for convenience of description, as an example of the annotation 106C, the information capable of specifying the position of the lesion region 110C in the rotated image 102C is shown, but this is merely an example. For example, the annotation 106C may include other types of information capable of specifying the lesion shown in the rotated image 102C, such as information capable of specifying a type of the lesion shown in the rotated image 102C.

As will be described in detail later, the radial ultrasound image 102D is an ultrasound image obtained by an actual radial ultrasound endoscopy. The lesion is shown in the radial ultrasound image 102D. That is, the radial ultrasound image 102D includes a lesion region 110D that is the portion corresponding to the lesion. An annotation 106D is assigned to the radial ultrasound image 102D. The annotation 106D is information capable of specifying a position of the lesion region 110D in the radial ultrasound image 102D (for example, information including a plurality of coordinates capable of specifying a position of a rectangular frame that circumscribes the lesion region 110D).

Here, for convenience of description, as an example of the annotation 106D, the information capable of specifying the position of the lesion region 110D in the radial ultrasound image 102D is shown, but this is merely an example. For example, the annotation 106D may include other types of information capable of specifying the lesion shown in the radial ultrasound image 102D, such as information capable of specifying a type of the lesion shown in the radial ultrasound image 102D.

As will be described in detail later, the virtual image 102E is a virtual ultrasound image that has an aspect imitating the radial ultrasound image. The lesion is shown in the virtual image 102E. That is, the virtual image 102E includes a lesion region 110E that is the portion corresponding to the lesion. An annotation 106E is assigned to the virtual image 102E. The annotation 106E is information capable of specifying a position of the lesion region 110E in the virtual image 102E (for example, information including a plurality of coordinates capable of specifying a position of a rectangular frame that circumscribes the lesion region 110E).

Here, for convenience of description, as an example of the annotation 106E, the information capable of specifying the position of the lesion region 110E in the virtual image 102E is shown, but this is merely an example. For example, the annotation 106E may include other types of information capable of specifying the lesion shown in the virtual image 102E, such as information capable of specifying a type of the lesion shown in the virtual image 102E.

It should be noted that, hereinafter, for convenience of description, in a case in which it is not necessary to distinguish among the circular image 102A, the scale-adjusted image 102B, the rotated image 102C, the radial ultrasound image 102D, and the virtual image 102E, the circular image 102A, the scale-adjusted image 102B, the rotated image 102C, the radial ultrasound image 102D, and the virtual image 102E are referred to as a “medical image 102”. In addition, hereinafter, for convenience of description, in a case in which it is not necessary to distinguish among the annotations 106A to 106E, the annotations 106A to 106E will be referred to as an “annotation 106”. In addition, hereinafter, for convenience of description, in a case in which it is not necessary to distinguish among the lesion regions 110A to 110E, the lesion regions 110A to 110E will be referred to as a “lesion region 110”. The annotation 106 is an example of an “annotation” according to the technology of the present disclosure.

The acquisition unit 92A acquires the plurality of medical images 102 from the NVM 96. For example, the acquisition unit 92A acquires the medical image 102 that has not yet been used to train the model 98 from the NVM 96 one frame at a time. The learning execution unit 92B trains the model 98 using the medical image 102 acquired by the acquisition unit 92A. It should be noted that, hereinafter, for convenience of description, in order to facilitate understanding of the processing executed by the learning execution unit 92B, a part of the processing executed by the learning execution unit 92B in accordance with the model 98 will be described as processing actively executed by the model 98 as a main subject. That is, for convenience of description, the model 98 will be described as having a function of executing processing (for example, processing including image recognition processing) on input information (for example, information including an image) and outputting a processing result.

The acquisition unit 92A inputs a training image 112, which is a portion (that is, a body of the medical image 102) of the medical image 102 other than the annotation 106, to the model 98. In a case in which the training image 112 is input to the model 98, the model 98 predicts the position of the lesion region 110 and outputs a prediction result 116. The prediction result 116 is information capable of specifying a position predicted by the model 98 as the position of the lesion region 110 in the training image 112. Examples of the information capable of specifying the position predicted by the model 98 include information including a plurality of coordinates capable of specifying a position of a bounding box surrounding a region predicted as a region in which the lesion region 110 is present (that is, a position of a bounding box in the training image 112).

The learning execution unit 92B calculates an error between the prediction result 116 and the annotation 106 corresponding to the prediction result 116 (that is, the annotation 106 assigned to the training image 112 input to the model 98 for outputting the prediction result 116). Then, the learning execution unit 92B executes adjustment in accordance with the calculated error on the model 98. That is, the learning execution unit 92B adjusts a plurality of optimization variables (for example, a plurality of connection weights and a plurality of offset values) in the model 98 such that the calculated error is minimized.

The acquisition unit 92A and the learning execution unit 92B optimize the model 98 by repeatedly executing learning processing, which is a series of processing of inputting the training image 112 to the model 98, calculating the error, and adjusting the plurality of optimization variables, for each of the plurality of medical images 102 (for example, all the medical images 102) stored in the NVM 96. For example, the model 98 is optimized by adjusting the plurality of optimization variables in the model 98 such that the error is minimized, so that the trained model 78 is generated. That is, the data structure of the trained model 78 is obtained by training the model 98 using the plurality of medical images 102 to which the annotations 106 are assigned.

The learning execution unit 92B transmits the trained model 78 to the processing device 18 via the communication module 90 (see FIG. 4). In the processing device 18, the processor 62 receives the trained model 78 via the communication module 60 (see FIG. 3) to store the received trained model 78 in the NVM 66.

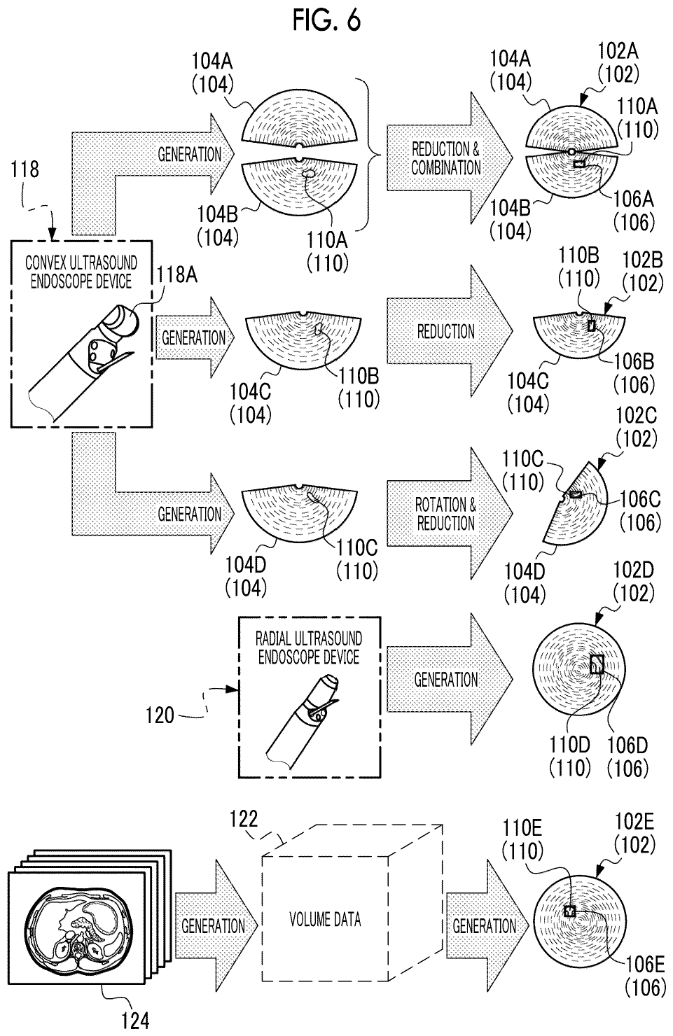

As shown in FIG. 6 as an example, the convex ultrasound image 104 is generated in the specific image mode by a convex ultrasound endoscope device 118 including a convex ultrasound probe 118A. There are a plurality of convex ultrasound images 104, and the convex ultrasound images 104 are obtained by the convex ultrasound endoscopies (for example, endoscopies using one or more convex ultrasound endoscope devices 118) executed on the plurality of subjects. The plurality of subjects are subjects different from the subject 22. It should be noted that the plurality of subjects may include the subject 22.

The circular image 102A is the image generated by combining the convex ultrasound images 104A and 104B. In the example shown in FIG. 6, the convex ultrasound image 104B includes the lesion region 110A. It should be noted that this is merely an example, and there may be a case in which the lesion is shown in the convex ultrasound image 104A or the lesion is shown in both of the convex ultrasound images 104A and 104B.

The convex ultrasound image 104A and the convex ultrasound image 104B are ultrasound images obtained by emitting the ultrasound in opposite directions via the ultrasound probe 118A, and have a linear symmetrical positional relationship. In addition, scales of the convex ultrasound images 104A and 104B are adjusted based on a scale of the radial ultrasound image 24. That is, since the scale of the radial ultrasound image 24 is smaller than the scales of the convex ultrasound images 104A and 104B, the convex ultrasound images 104A and 104B are reduced such that the scales of the convex ultrasound images 104A and 104B match the scale of the radial ultrasound image 24. Then, the reduced convex ultrasound images 104A and 104B are combined in a state in which the linear symmetrical positional relationship is maintained.

The circular image 102A is an image obtained by reducing the convex ultrasound images 104A and 104B in this manner and combining the reduced convex ultrasound images 104A and 104B while maintaining the linear symmetrical positional relationship. The annotation 106A corresponding to the lesion region 110A is assigned to the circular image 102A.

It should be noted that, in the example shown in FIG. 6, the convex ultrasound images 104A and 104B are disposed in a linear symmetrical manner in an up-down direction, but this is merely an example, and a pair of convex ultrasound images may be disposed in a linear symmetrical manner in a lateral direction or an oblique direction.

In addition, here, although the form example has described in which the pair of convex ultrasound images are disposed in a linear symmetrical manner and combined, this is merely an example. For example, convex ultrasound images of three or more frames obtained by executing convex scanning in different directions (for example, three or more directions) may be combined. In this case, in a case in which image regions overlap with each other between adjacent convex ultrasound images, an overlapping image region need only be removed from one of the adjacent convex ultrasound images to combine the adjacent convex ultrasound images.

In addition, here, although the form example has been described in which the pair of convex ultrasound images are combined after the scale is adjusted, this is merely an example, and the scale may be adjusted after the pair of convex ultrasound images are combined.

The scale-adjusted image 102B is an image obtained by adjusting a scale of the convex ultrasound image 104C including the lesion region 110B based on the scale of the radial ultrasound image 24. That is, since the scale of the radial ultrasound image 24 is smaller than the scale of the convex ultrasound image 104C, the convex ultrasound image 104C is reduced such that the scale of the convex ultrasound image 104C matches the scale of the radial ultrasound image 24. As described above, the image obtained by reducing the convex ultrasound image 104C is the scale-adjusted image 102B. The annotation 106B corresponding to the lesion region 110B is assigned to the scale-adjusted image 102B.

The rotated image 102C is an image obtained by rotating the convex ultrasound image 104D including the lesion region 110C. A rotation angle is, for example, a rotation angle designated in advance. There are a plurality of rotation angles, and the rotated image 102C is present for each rotation angle. The rotation angle is determined, for example, for each predetermined angle (for example, in units of 1 degree) in a range of 0 degrees or larger and smaller than 360 degrees. That is, the rotated images 102C are generated for the number of rotation angles for one convex ultrasound image 104D.

In addition, the rotated image 102C is also adjusted based on the scale of the radial ultrasound image 24, similarly to the circular image 102A and the scale-adjusted image 102B. That is, the convex ultrasound image 104D is reduced such that the scale of the convex ultrasound image 104D matches the scale of the radial ultrasound image 24. In this way, the image obtained by rotating the convex ultrasound image 104D by the designated rotation angle and reducing the rotated convex ultrasound image 104D is the rotated image 102C. The annotation 106C corresponding to the lesion region 110C is assigned to the rotated image 102C.

It should be noted that, here, although the form example has been described in which the scale is adjusted after the convex ultrasound image 104D is rotated, this is merely an example, and the rotated image 102C may be generated by rotating the convex ultrasound image 104D having the adjusted scale.

The radial ultrasound image 102D is a radial ultrasound image obtained by the radial ultrasound endoscopy on the subject (see FIG. 1) different from the subject 22. A plurality of radial ultrasound images 102D are present, and a plurality of radial ultrasound images 102D are generated for each of the plurality of subjects different from the subject 22. It should be noted that the plurality of subjects may include the subject 22.

In the radial ultrasound endoscopy, for example, a radial ultrasound endoscope device 120 is used. The radial ultrasound endoscope device 120 is preferably, for example, a device having the same specifications as the ultrasound endoscope device 12. In addition, it is preferable that the same parameters as various parameters set in the ultrasound endoscope device 12 are set in the radial ultrasound endoscope device 120 as parameters for controlling an image quality.

The radial ultrasound image 102D is a radial ultrasound image obtained in the specific image mode by the radial ultrasound endoscope device 120. In addition, the radial ultrasound image 102D is a radial ultrasound image obtained earlier than the radial ultrasound image 24 (see FIG. 1). That is, the radial ultrasound image 102D is a radial ultrasound image obtained from the radial ultrasound endoscopy executed earlier than the radial ultrasound endoscopy shown in FIG. 1. The observation target region shown in the radial ultrasound image 102D is anatomically the same region as the observation target region shown in the radial ultrasound image 24 (see FIG. 1). The radial ultrasound image 102D includes a lesion region 110D, and the annotation 106D corresponding to the lesion region 110D is assigned to the radial ultrasound image 102D.

The virtual image 102E is a virtual ultrasound image that is generated based on volume data 122 showing the subject and that has an aspect imitating the radial ultrasound image. A plurality of virtual images 102E are present and are generated for each volume data 122 indicating each of the plurality of subjects. It should be noted that the plurality of subjects may include the subject 22.

The volume data 122 is a three-dimensional image defined in a voxel by stacking a plurality of two-dimensional slice images 124 obtained by imaging the entire body or a part (for example, an abdomen) of the subject via a modality. A position of each voxel is specified by three-dimensional coordinates. Examples of the modality include a CT apparatus. The CT apparatus is merely an example, and other examples of the modality include an MRI apparatus and an ultrasound diagnostic device.

The virtual image 102E includes a lesion region 110E, and the annotation 106E corresponding to the lesion region 110E is assigned to the virtual image 102E.

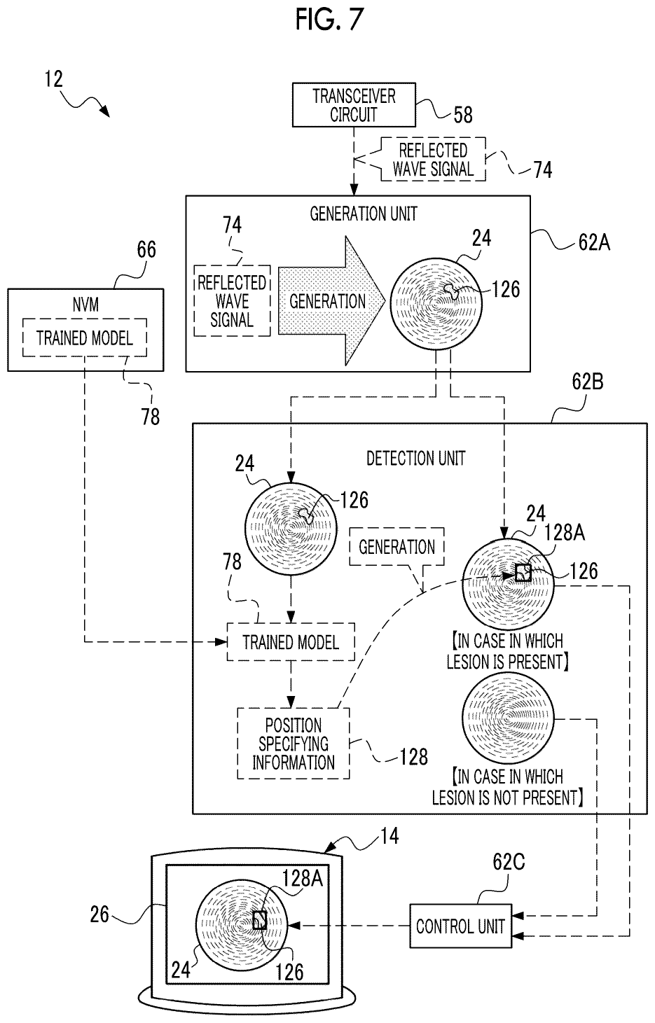

As shown in FIG. 7 as an example, in the ultrasound endoscope device 12, the generation unit 62A acquires the reflected wave signal 74 from the transceiver circuit 58 and generates the radial ultrasound image 24 based on the acquired reflected wave signal 74. The example shown in FIG. 7 shows an example in which the radial ultrasound image 24 including a lesion region 126, which is the portion corresponding to the lesion, is generated by the generation unit 62A, but the radial ultrasound image 24 in which the lesion is not shown may be generated by the generation unit 62A.

The detection unit 62B acquires the trained model 78 from the NVM 66. Then, the detection unit 62B detects the lesion from the radial ultrasound image 24 generated by the generation unit 62A in accordance with the acquired trained model 78. That is, the detection unit 62B determines the presence or absence of the lesion region 126 in the radial ultrasound image 24 in accordance with the trained model 78, and generates position specifying information 128 for specifying the position of the lesion region 126 (for example, information including a plurality of coordinates for specifying the position of the lesion region 126) in a case in which the lesion region 126 is present in the radial ultrasound image 24. It should be noted that, hereinafter, for convenience of description, in order to facilitate understanding of the processing executed by the detection unit 62B, a part of the processing executed by the detection unit 62B in accordance with the trained model 78 will be described as processing actively executed by the trained model 78 as a main subject. That is, for convenience of description, the trained model 78 will be described as having a function of executing processing (for example, processing including image recognition processing) on input information (for example, information including an image) and outputting a processing result.

The detection unit 62B inputs the radial ultrasound image 24 generated by the generation unit 62A to the trained model 78. In a case in which the radial ultrasound image 24 is input, the trained model 78 determines the presence or absence of the lesion region 126 in the radial ultrasound image 24. Here, in a case in which it is determined that the lesion region 126 is present in the radial ultrasound image 24 (that is, in a case in which the lesion shown in the radial ultrasound image 24 is detected), the trained model 78 outputs the position specifying information 128. The detection unit 62B generates a detection frame 128A based on the position specifying information 128 output from the trained model 78. The detection frame 128A is a rectangular frame corresponding to a bounding box (for example, a bounding box having a highest reliability score) used in a case in which the trained model 78 detects the lesion region 126 from the radial ultrasound image 24. That is, the detection frame 128A is a frame that surrounds the lesion region 126 detected by the trained model 78.

The detection unit 62B assigns the detection frame 128A to the radial ultrasound image 24 corresponding to the position specifying information 128 output from the trained model 78 (that is, the radial ultrasound image 24 input to the trained model 78 for outputting the position specifying information 128) in accordance with the position specifying information 128. That is, the detection unit 62B superimposes the detection frame 128A on the radial ultrasound image 24 corresponding to the position specifying information 128 output from the trained model 78 to surround the lesion region 126, thereby assigning the detection frame 128A to the radial ultrasound image 24. In a case in which the trained model 78 determines that the lesion region 126 is present in the radial ultrasound image 24, the detection unit 62B outputs the radial ultrasound image 24 to which the detection frame 128A is assigned, to the control unit 62C. In addition, in a case in which the trained model 78 determines that the lesion region 126 is not present in the radial ultrasound image 24, the detection unit 62B outputs the radial ultrasound image 24 to which the detection frame 128A is not assigned, to the control unit 62C.

The control unit 62C displays the radial ultrasound image 24 (that is, the radial ultrasound image 24 in which the detection result of the detection unit 62B is reflected) input from the detection unit 62B on the screen 26 of the display device 14. In a case in which the lesion is shown in the radial ultrasound image 24, the radial ultrasound image 24 to which the detection frame 128A surrounding the lesion region 126 is assigned (that is, the radial ultrasound image 24 on which the detection frame 128A is superimposed) is displayed on the screen 26. On the other hand, in a case in which the lesion is not shown in the radial ultrasound image 24, the radial ultrasound image 24 to which the detection frame 128A is not assigned (that is, the radial ultrasound image 24 output from the trained model 78) is displayed on the screen 26.

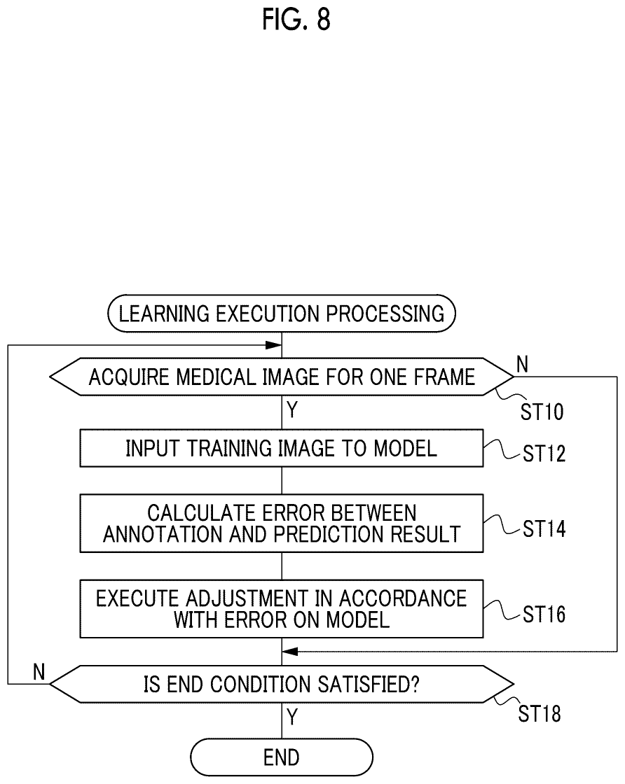

Next, an operation of the learning device 80 will be described with reference to FIG. 8.

FIG. 8 shows an example of a flow of the learning execution processing executed by the processor 92 of the learning device 80. The flow of the learning execution processing shown in FIG. 8 is an example of a “learning method” according to the technology of the present disclosure.

In the learning execution processing shown in FIG. 8, first, in step ST10, the acquisition unit 92A acquires the medical image 102 for one frame, which has not yet been used to train the model 98, from the NVM 96. After the processing of step ST10 is executed, the learning execution processing proceeds to step ST12.

In step ST12, the learning execution unit 92B inputs the training image 112 obtained from the medical image 102 acquired in step ST10 to the model 98. After the processing of step ST12 is executed, the learning execution processing proceeds to step ST14.

In step ST14, the learning execution unit 92B calculates the error between the annotation 106 assigned to the medical image 102 acquired in step ST10 and the prediction result 116 output from the model 98 by executing the processing of step ST12. After the processing of step ST14 is executed, the learning execution processing proceeds to step ST16.

In step ST16, the learning execution unit 92B executes the adjustment in accordance with the error calculated in step ST14 on the model 98. After the processing of step ST14 is executed, the learning execution processing proceeds to step ST18.

In step ST18, the learning execution unit 92B determines whether or not a condition for ending the learning execution processing (hereinafter, referred to as a “learning end condition”) is satisfied. A first example of the learning end condition is a condition in which all of the medical images 102 in the NVM 96 are used to train the model 98. A second example of the learning end condition is a condition in which an instruction to end the learning execution processing is received by the reception device 86.

In step ST18, in a case in which the learning end condition is not satisfied, a negative determination is made, and the learning execution processing proceeds to step ST10. In step ST18, in a case in which the learning end condition is satisfied, an affirmative determination is made, and the learning execution processing ends.

The model 98 is optimized by repeatedly executing the processing of step ST10 to the processing of step ST18, so that the trained model 78 is generated. The trained model 78 generated in this way is stored in the NVM 66 (see FIGS. 3 and 5).

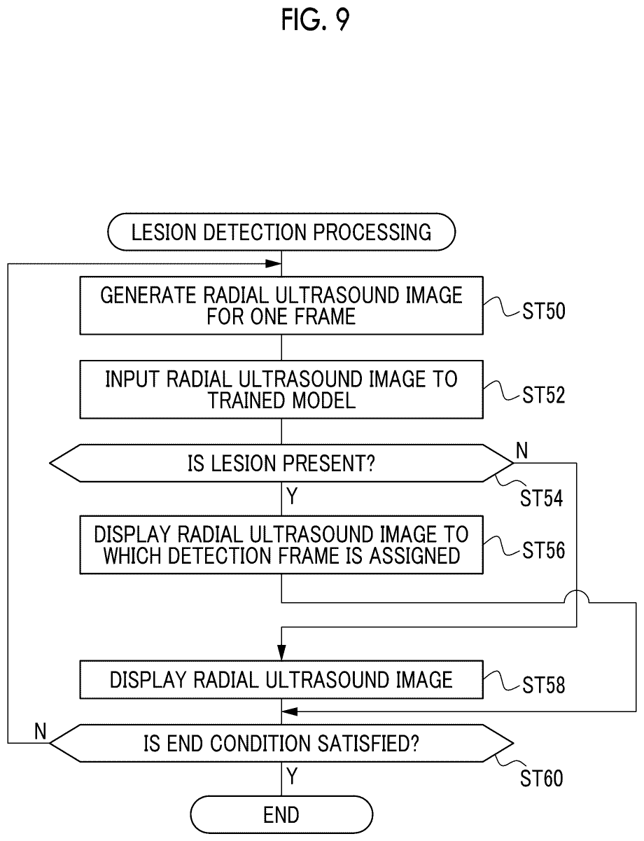

Next, an operation of the endoscope system 10 will be described with reference to FIG. 9.

FIG. 9 shows an example of a flow of the lesion detection processing executed by the processor 62 of the processing device 18.

In the lesion detection processing shown in FIG. 9, first, in step ST50, the generation unit 62A acquires the reflected wave signal 74 from the transceiver circuit 58, and generates the radial ultrasound image 24 for one frame based on the acquired reflected wave signal 74. After the processing of step ST50 is executed, the lesion detection processing proceeds to step ST52.

In step ST52, the detection unit 62B inputs the radial ultrasound image 24 generated in step ST50 to the trained model 78. After the processing of step ST52 is executed, the lesion detection processing proceeds to step ST54.

In step ST54, the detection unit 62B determines whether or not the lesion is shown in the radial ultrasound image 24 input to the trained model 78 in step ST52, by using the trained model 78. In a case in which the lesion is shown in the radial ultrasound image 24, the trained model 78 outputs the position specifying information 128.

In step ST54, in a case in which the lesion is not shown in the radial ultrasound image 24, a negative determination is made, and the lesion detection processing proceeds to step ST58. In step ST54, in a case in which the lesion is shown in the radial ultrasound image 24, an affirmative determination is made, and the lesion detection processing proceeds to step ST56.

In a case in which an affirmative determination is made in step ST54, the detection unit 62B generates the detection frame 128A based on the position specifying information 128 output from the trained model 78, and superimposes the detection frame 128A on the radial ultrasound image 24 generated in step ST50 to surround the lesion region 126. Then, in step ST56, the control unit 62C displays the radial ultrasound image 24 in which the lesion region 126 is surrounded by the detection frame 128A on the screen 26 of the display device 14. Since the lesion region 126 in the radial ultrasound image 24 is surrounded by the detection frame 128A, the doctor 20 can visually understand the position at which the lesion is shown in the radial ultrasound image 24. After the processing of step ST56 is executed, the lesion detection processing proceeds to step ST60.

In step ST58, the control unit 62C displays the radial ultrasound image 24 generated in step ST50 on the screen 26 of the display device 14. In this case, since the detection frame 128A is not assigned to the radial ultrasound image 24, the doctor 20 can visually recognize that the lesion is not shown in the radial ultrasound image 24. After the processing of step ST58 is executed, the lesion detection processing proceeds to step ST60.

In step ST60, the control unit 62C determines whether or not a condition for ending the lesion detection processing (hereinafter, referred to as a “lesion detection end condition”) is satisfied. Examples of the lesion detection end condition include a condition in which an instruction to end the lesion detection processing is received by the reception device 52. In a case in which the lesion detection end condition is not satisfied in step ST60, a negative determination is made, and the lesion detection processing proceeds to step ST50. In step ST60, in a case in which the lesion detection end condition is satisfied, an affirmative determination is made, and the lesion detection processing ends.

As described above, the annotation 106 is assigned to each of the plurality of medical images 102 stored in the NVM 96 of the learning device 80. The annotation 106 is the information capable of specifying a position of the lesion region 110 in the medical image 102. The medical image 102 is the image that is generated based on at least one convex ultrasound image 104 and that has an aspect imitating at least a part of the radial ultrasound image. Then, the plurality of medical images 102 formed in this way are used to train the model 98. That is, the model 98 is optimized by repeatedly executing the learning processing of inputting the training image 112, which is a main body of the medical image 102, to the model 98, calculating the error, and adjusting the plurality of optimization variables for each of the plurality of medical images 102 stored in the NVM 96. The trained model 78 is generated by optimizing the model 98, and the trained model 78 is used to detect the lesion shown in the radial ultrasound image 24. As described above, with the learning device 80, it is possible to obtain the trained model 78 that contributes to specifying the lesion shown in the radial ultrasound image 24 without training the model 98 only using the radial ultrasound image (for example, the radial ultrasound image 102D shown in FIGS. 5 and 6).

The plurality of medical images 102 stored in the NVM 96 of the learning device 80 include the circular image 102A. The circular image 102A is the image generated by combining the convex ultrasound images 104A and 104B. Since the outer shape of the radial ultrasound image 24 is also a circular shape, the trained model 78 obtained by training the model 98 using the circular image 102A having the same outer shape can contribute to high-accuracy specification of the lesion shown in the radial ultrasound image 24.

The scales of the convex ultrasound images 104A and 104B, which are the bases of the circular image 102A, are different from the scale of the radial ultrasound image 24. Therefore, the learning device 80 generates the circular image 102A by combining the convex ultrasound images 104A and 104B of which the scale is adjusted based on the scale of the radial ultrasound image 24 (for example, the convex ultrasound images 104A and 104B of which the scales match the scale of the radial ultrasound image 24), and trains the model 98. As a result, it is possible to improve the accuracy of specifying the lesion from the radial ultrasound image 24 in accordance with the trained model 78, as compared to a case in which the model 98 is trained using the circular image 102A having the same scale as the scales of the convex ultrasound images 104A and 104B.

The plurality of different circular images 102A are stored in the NVM 96 of the learning device 80 in advance. Then, the processor 92 acquires the circular image 102A from the NVM 96 and trains the model 98 using the acquired circular image 102A. Therefore, the learning device 80 can train the model 98 using the circular image 102A without causing the processor 92 to generate the circular image 102A each time the model 98 is trained.

The number of orientations in which the ultrasound is emitted by the radial scanning is larger than the number of orientations in which the ultrasound is emitted by the convex scanning. Therefore, in order to enable the detection of the lesion in a region corresponding to the orientation in which the ultrasound wave is not emitted in the convex scanning, the plurality of medical images 102 stored in the NVM 96 of the learning device 80 include the rotated image 102C. The rotated image 102C is the image obtained by rotating the convex ultrasound image 104D. Therefore, by training the model 98 using the rotated image 102C, even in a case in which the lesion is shown at various positions in the radial ultrasound image 24 used for the diagnosis, it is possible to improve the accuracy of specifying the lesion from the radial ultrasound image 24 in accordance with the trained model 78.

The scale of the convex ultrasound image 104D, which is the basis of the rotated image 102C, is different from the scale of the radial ultrasound image 24. Therefore, the learning device 80 generates the rotated image 102C by adjusting the scale of the rotated convex ultrasound image 104D based on the scale of the radial ultrasound image 24 (for example, by matching the scale of the rotated convex ultrasound image 104D with the scale of the radial ultrasound image 24), and trains the model 98 using the generated rotated image 102C. As a result, it is possible to improve the accuracy of specifying the lesion from the radial ultrasound image 24 in accordance with the trained model 78, as compared to a case in which the model 98 is trained using the rotated image 102C having the same scale as the scale of the convex ultrasound image 104D.

The plurality of different rotated images 102C are stored in the NVM 96 of the learning device 80 in advance. Then, the processor 92 acquires the rotated image 102C from the NVM 96 and trains the model 98 using the acquired rotated image 102C. Therefore, the learning device 80 can train the model 98 using the rotated image 102C without causing the processor 92 to generate the rotated image 102C each time the model 98 is trained.

The scale of the convex ultrasound image 104C is different from the scale of the radial ultrasound image 24. Therefore, the learning device 80 generates the scale-adjusted image 102B by adjusting the scale of the convex ultrasound image 104C based on the scale of the radial ultrasound image 24 (for example, by matching the scale of the convex ultrasound image 104C to the scale of the radial ultrasound image 24), and trains the model 98 using the scale-adjusted image 102B. As a result, it is possible to improve the accuracy of specifying the lesion from the radial ultrasound image 24 in accordance with the trained model 78, as compared to a case in which the model 98 is trained using the convex ultrasound image 104C without adjusting the scale of the convex ultrasound image 104C.

The plurality of different scale-adjusted images 102B are stored in the NVM 96 of the learning device 80 in advance. Then, the processor 92 acquires the scale-adjusted image 102B from the NVM 96 and trains the model 98 using the acquired scale-adjusted image 102B. Therefore, the learning device 80 can train the model 98 using the scale-adjusted image 102B without causing the processor 92 to generate the scale-adjusted image 102B each time the model 98 is trained.

In the learning device 80, the processor 92 acquires the radial ultrasound image 102D from the NVM 96 and trains the model 98 using the acquired radial ultrasound image 102D. Therefore, it is possible to obtain the trained model 78 that contributes to high-accuracy detection of the lesion from the radial ultrasound image 24, as compared to a case in which the model 98 is not trained using the radial ultrasound image 102D (that is, as compared to a case in which the model 98 is trained using only the image generated based on the convex ultrasound image 104).

The virtual image 102E is stored in the NVM 96 of the learning device 80. The virtual image 102E is a virtual ultrasound image that is generated based on the volume data 122 and that has an aspect imitating the radial ultrasound image. The processor 92 acquires the virtual image 102E from the NVM 96 and trains the model 98 using the acquired virtual image 102E. Therefore, even in a case in which the number of actual ultrasound images (for example, the convex ultrasound image 104 and/or the radial ultrasound image 102D) used to train the model 98 is insufficient, the insufficient number can be made up for by the virtual image 102E.

### First Modification Example

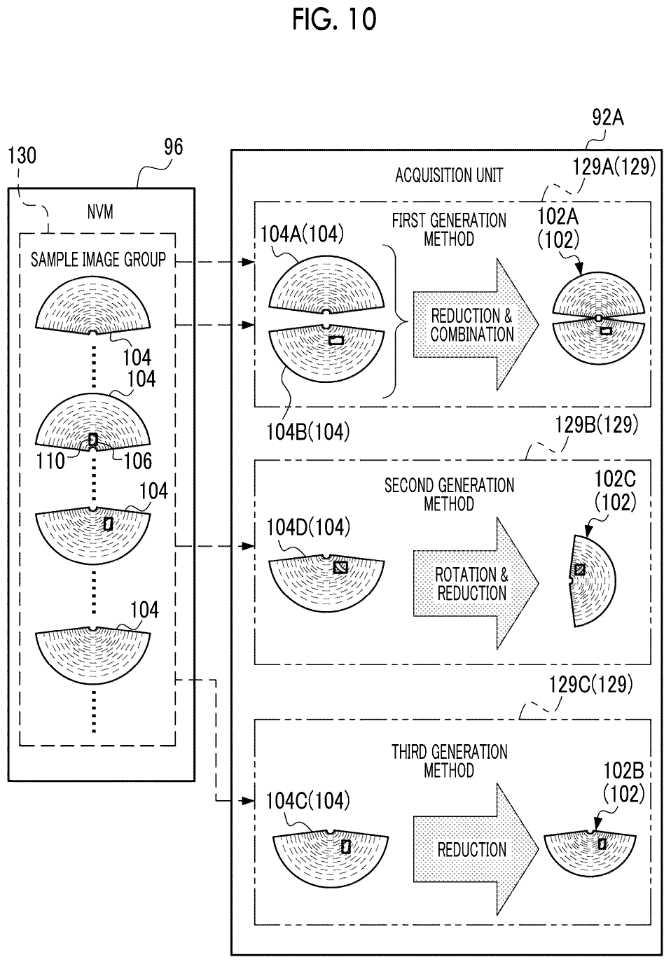

In the embodiment described above, the form example has been described in which the plurality of medical images 102 stored in the NVM 96 in advance are acquired by the acquisition unit 92A, and the model 98 is trained using the plurality of acquired medical images 102, but the technology of the present disclosure is implementable even in a case in which the plurality of medical images 102 are not stored in the NVM 96 in advance. For example, as shown in FIG. 10, the acquisition unit 92A may acquire the medical image 102 by randomly selecting one generation method 129 from among a plurality of generation methods 129 for generating the medical image 102 based on at least one convex ultrasound image 104, and generating the medical image 102 in accordance with the selected generation method 129. In this case, the model 98 need only be trained using the medical image 102 acquired by the acquisition unit 92A in the same manner as in the embodiment described above.

In the example shown in FIG. 10, as an example of the plurality of generation methods 129, a first generation method 129A, a second generation method 129B, and a third generation method 129C are shown. The first generation method 129A is a method including generating the circular image 102A as the medical image 102 by combining the plurality of convex ultrasound images 104, and adjusting the scale of the circular image 102A based on the scale of the radial ultrasound image 24. The second generation method 129B is a method including generating the rotated image 102C in which the convex ultrasound image 104 is rotated, as the medical image 102, and adjusting the scale of the rotated image 102C based on the scale of the radial ultrasound image 24. The third generation method 129C is a method including generating the scale-adjusted image 102B as the medical image 102 by adjusting the scale of the convex ultrasound image 104 based on the scale of the radial ultrasound image 24.

The NVM 96 stores a sample image group 130. The sample image group 130 consists of the plurality of convex ultrasound images 104. The plurality of convex ultrasound images 104 constituting the sample image group 130 are images that are the basis of the medical images 102 (for example, the circular image 102A, the scale-adjusted image 102B, and the rotated image 102C). That is, the sample image group 130 includes the convex ultrasound images 104A, 104B, 104C, and 104D shown in FIG. 6. In addition, the plurality of convex ultrasound images 104 constituting the sample image group 130 include the convex ultrasound image 104 including the lesion region 110 and the annotation 106 corresponding to the lesion region 110.

The acquisition unit 92A randomly selects one generation method 129 from among the first generation method 129A, the second generation method 129B, and the third generation method 129C, and acquires at least one convex ultrasound image 104 used in the selected generation method 129 from the sample image group 130.

In a case in which the first generation method 129A is selected, the acquisition unit 92A acquires the convex ultrasound images 104A and 104B from the sample image group 130. The acquisition unit 92A acquires the convex ultrasound images 104A and 104B having different combinations from the sample image group 130 each time the first generation method 129A is selected. In this case, at least one of the convex ultrasound image 104A or the convex ultrasound image 104B includes the lesion region 110. That is, the annotation 106 is assigned to at least one of the convex ultrasound image 104A or the convex ultrasound image 104B. The acquisition unit 92A generates the circular image 102A in the same manner as in the example shown in FIG. 6, by using the convex ultrasound images 104A and 104B acquired from the sample image group 130.

In a case in which the second generation method 129B is selected, the acquisition unit 92A acquires the convex ultrasound image 104D from the sample image group 130. Each time the second generation method 129B is selected, the acquisition unit 92A randomly acquires the convex ultrasound image 104D from the sample image group 130, and randomly determines the rotation angle for rotating the convex ultrasound image 104D. Then, the acquisition unit 92A generates the rotated image 102C in the same manner as in the example shown in FIG. 6, by using the convex ultrasound image 104D acquired from the sample image group 130.

In a case in which the third generation method 129C is selected, the acquisition unit 92A acquires the convex ultrasound image 104C that has not yet been used to generate the scale-adjusted image 102B from the sample image group 130. Then, the acquisition unit 92A generates the scale-adjusted image 102B in the same manner as in the example shown in FIG. 6, by using the convex ultrasound image 104C acquired from the sample image group 130.

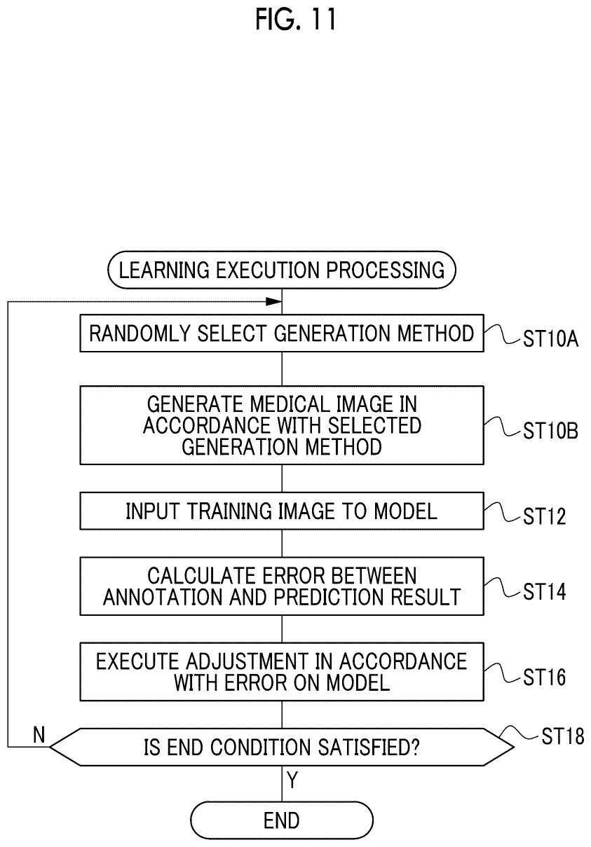

FIG. 11 shows an example of a flow of learning execution processing according to the first modification example. The flowchart shown in FIG. 11 is different from the flowchart shown in FIG. 8 in that processing of step ST10A and processing of step ST10B are provided instead of the processing of step ST10.

In the learning execution processing shown in FIG. 11, in step ST10A, the acquisition unit 92A selects one generation method 129 from among the plurality of generation methods 129 (for example, the first to third generation methods 129A to 129C). After the processing of step ST10A is executed, the learning execution processing proceeds to step ST10B.

In step ST10B, the acquisition unit 92A generates the medical image 102 (for example, the circular image 102A, the scale-adjusted image 102B, or the rotated image 102C) according to the generation method 129 (for example, any one of the first generation method 129A, the second generation method 129B, or the third generation method 129C) selected in step ST10B.

In step ST12, the training image 112 (see FIG. 5) obtained from the medical image 102 generated in step ST10B is input to the model 98. It should be noted that, in the model 98, the radial ultrasound image 102D and/or the virtual image 102E may be input in the same manner as in the embodiment described above.

As described above, in the first modification example, the acquisition unit 92A randomly selects one generation method 129 from among the plurality of generation methods 129, and generates the medical image 102 in accordance with the selected generation method 129 to acquire the medical image 102. Then, the model 98 is trained using the medical image 102 acquired by the acquisition unit 92A in the same manner as in the embodiment described above. Therefore, it is possible to suppress bias in the training of the model 98, as compared to a case in which the model 98 is trained using the medical image 102 generated by only one generation method 129 at all times. Since it is not necessary to store the medical image 102 in advance in the memory such as the NVM 96, it is possible to prevent the memory from running out of capacity.

The plurality of generation methods 129 include the first generation method 129A, the second generation method 129B, and the third generation method 129C. The first generation method 129A is the method including generating the circular image 102A, the second generation method 129B is the method including generating the rotated image 102C, and the third generation method 129C is the method including generating the scale-adjusted image 102B. The first generation method 129A, the second generation method 129B, and the third generation method 129C are randomly selected by the acquisition unit 92A, and the circular image 102A, the scale-adjusted image 102B, or the rotated image 102C is randomly generated by the selected generation method 129. Therefore, the model 98 can be trained randomly using the circular image 102A, the scale-adjusted image 102B, and the rotated image 102C. As a result, it is possible to suppress the bias in the training of the model 98, as compared to a case in which the model 98 is trained using the medical image 102 generated by only one generation method 129 at all times. Since it is not necessary to store the medical image 102 in advance in the memory such as the NVM 96, it is possible to prevent the memory from running out of capacity.

In the first generation method 129A, the scale of the circular image 102A is adjusted based on the scale of the radial ultrasound image 24, as in the example shown in FIG. 6. For example, the circular image 102A is reduced such that the scale of the circular image 102A matches the scale of the radial ultrasound image 24. Therefore, it is possible to improve the accuracy of specifying the lesion from the radial ultrasound image 24 in accordance with the trained model 78, as compared to a case in which the scale of the circular image 102A is the same as the scales of the convex ultrasound images 104A and 104B.

In the third generation method 129C, the scale of the rotated image 102C is adjusted based on the scale of the radial ultrasound image 24, as in the example shown in FIG. 6. For example, the rotated image 102C is reduced such that the scale of the rotated image 102C matches the scale of the radial ultrasound image 24. Therefore, it is possible to improve the accuracy of specifying the lesion from the radial ultrasound image 24 in accordance with the trained model 78, as compared to a case in which the scale of the rotated image 102C is the same as the scale of the convex ultrasound image 104D.

In the first modification example, the first generation method 129A, the second generation method 129B, and the third generation method 129C are described as examples, but the plurality of generation methods 129 may include a generation method 129 other than the first generation method 129A, the second generation method 129B, and the third generation method 129C. Examples of the generation method 129 other than the first generation method 129A, the second generation method 129B, and the third generation method 129C include a method for generating an image (hereinafter, referred to as a “partial image”) corresponding to a partial region in the convex ultrasound image 104 as the medical image 102. Examples of the image corresponding to the partial region include a divided image including the lesion region 110 among a plurality of divided images obtained by dividing the convex ultrasound image 104. Another example of the generation method 129 includes a method for generating an image (hereinafter, referred to as a “rotated circular image”) in which the circular image 102A is rotated.

It should be noted that the partial image and/or the rotated circular image may be stored in advance as the medical image 102 in the NVM 96 as shown in FIG. 5. In this case, the partial image and/or the rotated circular image is acquired from the NVM 96 by the acquisition unit 92A and is used to train the model 98, similarly to the other medical images 102.

### Other Modification Examples

In the embodiment described above, the form example has been described in which the radial ultrasound image 24 and the detection frame 128A, which are generated by the processing device 18, are displayed on the screen 26 of the display device 14, but the radial ultrasound image 24 to which the detection frame 128A is assigned may be transmitted to various devices such as a server, a PC, and/or a tablet terminal and stored in memories of the various devices. In addition, the radial ultrasound image 24 to which the detection frame 128A is assigned may be recorded in a report. In addition, the position specifying information 128 may also be stored in the memories of the various devices or may be recorded in the report. It is preferable that the radial ultrasound image 24, the detection frame 128A, and/or the position specifying information 128 is stored in the memories or recorded in the report for each subject 22.

In the embodiment described above, although the form example has been described in which the convex ultrasound image 104 is reduced such that the scale of the convex ultrasound image 104 matches the scale of the radial ultrasound image 24, in a case in which the scale of the radial ultrasound image 24 is larger than the scale of the convex ultrasound image 104, the convex ultrasound image 104 may be enlarged such that the scale of the convex ultrasound image 104 matches the scale of the radial ultrasound image 24.

In the embodiment described above, the form example has been described in which the lesion detection processing is executed by the processing device 18 and the learning execution processing is executed by the learning device 80, but the technology of the present disclosure is not limited to this. The lesion detection processing may be executed by the processing device 18 and at least one device provided outside the processing device 18, or may be executed only by at least one device (for example, an auxiliary processing device that is connected to the processing device 18 and that is used to expand the functions of the processing device 18) provided outside the processing device 18. In addition, the learning execution processing may be executed by the learning device 80 and the at least one device provided outside the learning device 80, or may be executed only by the at least one device provided outside the learning device 80.

Examples of the at least one device provided outside the processing device 18 and the at least one device provided outside the learning device 80 include a server. The server may be implemented by cloud computing. The cloud computing is merely an example, and network computing, such as fog computing, edge computing, or grid computing, may be used. In addition, the server described as the at least one device provided outside the processing device 18 and the at least one device provided outside the learning device 80 is merely an example, and may be at least one PC and/or at least one mainframe instead of the server, or may be at least one server, at least one PC, and/or at least one mainframe.

In the embodiment described above, the form example has been described in which the radial ultrasound image 24 on which the detection frame 128A is superimposed is displayed on the screen 26 of the display device 14, but this is merely an example. For example, the radial ultrasound image 24 on which the detection frame 128A is superimposed and the radial ultrasound image 24 on which the detection frame 128A is not superimposed (that is, the radial ultrasound image 24 in which the result of detecting the lesion region 126 is not visualized) may be displayed on separate screens.

In the embodiment described above, the presence or absence of the lesion and the position of the lesion are visually recognized by the doctor 20 by displaying the detection frame 128A in a state of being superimposed on the radial ultrasound image 24, but notification of the presence or absence of the lesion and the position of the lesion may be issued by using a notification method (for example, a text image, sound information, or the like) other than the detection frame 128A.

In the embodiment described above, the doctor 20 perceives the presence or absence of the lesion and the position of the lesion, but the doctor 20 may perceive the type of the lesion and/or the degree of progress of the lesion. In this case, the medical image 102 need only be used as the training data for the training of the model 98 in a state in which the annotation 106 includes the information capable of specifying the type of the lesion and/or the degree of progress of the lesion.

In the embodiment described above, the form example has been described in which the NVM 96 stores the learning execution program 100, but the technology of the present disclosure is not limited to this. For example, the learning execution program 100 may be stored in a portable storage medium, such as an SSD or a USB memory. The storage medium is a non-transitory computer-readable storage medium. The learning execution program 100 stored in the storage medium is installed in the computer 82. The processor 92 executes the learning execution processing in accordance with the learning execution program 100.

In the embodiment described above, the form example has been described in which the NVM 66 stores the lesion detection program 76, but the technology of the present disclosure is not limited to this. For example, the lesion detection program 76 may be stored in a portable storage medium, such as an SSD or a USB memory. The storage medium is a non-transitory computer-readable storage medium. The lesion detection program 76 stored in the storage medium is installed in the computer 54. The processor 62 executes the lesion detection processing in accordance with the lesion detection program 76.

In the embodiment described above, the computers 54 and 82 are described as examples, but the technology of the present disclosure is not limited to this, and a device including an ASIC, an FPGA, and/or a PLD may be applied instead of the computers 54 and/or 82. A combination of a hardware configuration and a software configuration may be used instead of the computers 54 and/or 82.

The following various processors can be used as a hardware resource for executing various types of processing (that is, learning execution processing and lesion detection processing) described in the embodiment described above. Examples of the processor include a processor as a general-purpose processor that executes software, that is, a program, to function as the hardware resource executing the various types of processing. Examples of the processor also include a dedicated electronic circuit as a processor having a dedicated circuit configuration designed to execute specific processing, such as an FPGA, a PLD, or an ASIC. Any processor has a memory built in or connected to it, and any processor executes the various types of processing by using the memory.

The hardware resource for executing the various types of processing may be configured by one of the various processors or by a combination of two or more processors of the same type or different types (for example, a combination of a plurality of FPGAs or a combination of a processor and an FPGA). Further, the hardware resource for executing the various types of processing may be one processor.

A first example of the configuration in which the hardware resource is configured by one processor is an aspect in which one processor is configured by a combination of one or more processors and software, and this processor functions as the hardware resource for executing the various types of processing. As a second example, as typified by an SoC or the like, there is a form in which a processor that implements all functions of a system including a plurality of hardware resources executing the various types of processing with one IC chip is used. As described above, the various types of processing are implemented by using one or more of the various processors as the hardware resource.

Further, specifically, an electronic circuit obtained by combining circuit elements, such as semiconductor elements, can be used as the hardware structure of the various processors. The various types of processing are merely examples. Therefore, it goes without saying that unnecessary steps may be deleted, new steps may be added, or the processing order may be changed within a range that does not deviate from the gist.

The above-described contents and the above-shown contents are the detailed description of the parts according to the technology of the present disclosure, and are merely examples of the technology of the present disclosure. For example, the description of the configuration, the function, the operation, and the effect is the description of examples of the configuration, the function, the operation, and the effect of the parts according to the technology of the present disclosure. Accordingly, it goes without saying that unnecessary parts may be deleted, new elements may be added, or replacements may be made with respect to the above-described contents and the above-shown contents within a range that does not deviate from the gist of the technology of the present disclosure. In addition, in order to avoid complications and facilitate understanding of the parts according to the technology of the present disclosure, the description of common technical knowledge or the like, which does not particularly require the description for enabling the implementation of the technology of the present disclosure, is omitted in the above-described contents and the above-shown contents.

In the present specification, “A and/or B” is synonymous with “at least one of A or B”. That is, “A and/or B” may mean only A, only B, or a combination of A and B. In the present specification, the same concept as “A and/or B” also applies to a case in which three or more matters are expressed by association with “and/or”.

All of the documents, the patent applications, and the technical standards described in the present specification are incorporated into the present specification by reference to the same extent as in a case in which the individual documents, patent applications, and technical standards are specifically and individually stated to be described by reference.

What is claimed is:

1. A learning device comprising:
a first processor,
wherein the first processor is configured to:
acquire a plurality of medical images to which annotations for specifying a lesion are assigned, and
train a model using the plurality of acquired medical images, and
the medical image is an image that is generated based on at least one convex ultrasound image and that has an aspect imitating at least a part of a radial ultrasound image.

2. The learning device according to claim 1,
wherein the plurality of medical images include a circular image generated by combining a plurality of the convex ultrasound images.

3. The learning device according to claim 2,
wherein a scale of the circular image is adjusted based on a scale of the radial ultrasound image.

4. The learning device according to claim 2,
wherein the circular image is stored in a first memory in advance, and
the first processor is configured to:
acquire the circular image from the first memory, and
train the model using the acquired circular image.

5. The learning device according to claim 1,
wherein the plurality of medical images include a rotated image obtained by rotating the convex ultrasound image.

6. The learning device according to claim 5,
wherein a scale of the rotated image is adjusted based on a scale of the radial ultrasound image.

7. The learning device according to claim 5,
wherein the rotated image is stored in a second memory in advance, and
the first processor is configured to:
acquire the rotated image from the second memory, and
train the model using the acquired rotated image.

8. The learning device according to claim 1,
wherein the plurality of medical images include a scale-adjusted image obtained by adjusting a scale of the convex ultrasound image based on a scale of the radial ultrasound image.

9. The learning device according to claim 8,
wherein the scale-adjusted image is stored in a third memory in advance, and
the first processor is configured to:
acquire the scale-adjusted image from the third memory, and
train the model using the acquired scale-adjusted image.

10. The learning device according to claim 1,
wherein the first processor is configured to:
randomly select one generation method from among a plurality of generation methods for generating the medical image based on the at least one convex ultrasound image,
acquire the medical image by generating the medical image in accordance with the selected generation method, and
train the model using the acquired medical image.

11. The learning device according to claim 10,
wherein the plurality of generation methods include a first generation method, a second generation method, and a third generation method,
the first generation method includes generating a circular image as the medical image by combining a plurality of the convex ultrasound images,
the second generation method includes generating a rotated image in which the convex ultrasound image is rotated, as the medical image, and
the third generation method includes generating a scale-adjusted image as the medical image by adjusting a scale of the convex ultrasound image based on a scale of the radial ultrasound image.

12. The learning device according to claim 11,
wherein the first generation method includes adjusting a scale of the circular image based on the scale of the radial ultrasound image.

13. The learning device according to claim 11,
wherein the second generation method includes adjusting a scale of the rotated image based on the scale of the radial ultrasound image.

14. The learning device according to claim 1,
wherein the first processor is configured to:
acquire at least one first ultrasound image obtained by a first radial ultrasound endoscope, and
train the model using the acquired first ultrasound image.

15. The learning device according to claim 1,
wherein the first processor is configured to:
acquire a virtual ultrasound image that is generated based on volume data showing a subject and that has an aspect imitating at least a part of the radial ultrasound image, and
train the model using the acquired virtual ultrasound image.

16. A trained model obtained by training the model using the plurality of medical images via the learning device according to claim 1.

17. A trained model comprising:
a data structure used for processing of specifying a lesion from a second ultrasound image obtained by a second radial ultrasound endoscope, wherein:
the data structure is obtained by training a model using a plurality of medical images to which annotations for specifying the lesion are assigned, and
the medical image is an image that is generated based on at least one convex ultrasound image and that has an aspect imitating at least a part of a radial ultrasound image.

18. A medical diagnostic device comprising:
the trained model according to claim 17; and
a second processor,
wherein the second processor is configured to:
acquire a third ultrasound image obtained by a third radial ultrasound endoscope, and
detect a portion corresponding to the lesion from the acquired third ultrasound image in accordance with the trained model.

19. An ultrasound endoscope device comprising:
the trained model according to claim 17;
a fourth radial ultrasound endoscope; and
a third processor,
wherein the third processor is configured to:
acquire a fourth ultrasound image obtained by the fourth radial ultrasound endoscope, and
detect a portion corresponding to the lesion from the acquired fourth ultrasound image in accordance with the trained model.

20. A learning method comprising:
acquiring a plurality of medical images to which annotations for specifying a lesion are assigned; and
training a model using the plurality of acquired medical images,
wherein the medical image is an image that is generated based on at least one convex ultrasound image and that has an aspect imitating at least a part of a radial ultrasound image.

21. A non-transitory computer-readable storage medium storing a program for executable by a computer to perform a process comprising:
acquiring a plurality of medical images to which annotations for specifying a lesion are assigned; and
training a model using the plurality of acquired medical images,
wherein the medical image is an image that is generated based on at least one convex ultrasound image and that has an aspect imitating at least a part of a radial ultrasound image.
